(19) United States
(12) Patent Application Publication
(10) Pub. No.: US 2024/0293183 A1
(43) Pub. Date: Sep. 5, 2024

(54) SYSTEMS, METHODS, AND DEVICES FOR AUGMENTED DENTAL IMPLANT SURGERY USING KINEMATIC DATA

(71) Applicant: MODJAW, Villeurbanne (FR)

(72) Inventors: Maxime Jaisson, Porte-de-Savoie (FR); Antoine Jules Rodrigue, Lyon (FR)

(21) Appl. No.: 18/572,620

(22) PCT Filed: Jun. 21, 2022
(86) PCT No.: PCT/IB2022/000368

§ 371 Date: Dec. 20, 2023

Related U.S. Application Data
(60) Provisional application No. 63/213,607, filed on Jun. 22, 2021.

(51) Int. Cl. A61B 34/10; A61B 34/20; G06T 7/00; G06T 17/00

(52) U.S. Cl. CPC A61B 34/10; G06T 7/00; G06T 17/00; A61B 2034/105; A61B 2034/108; A61B 2034/2048; G06T 2207/30036

(57) ABSTRACT

Systems and methods for modeling and planning a dental implant procedure are provided. A method for modeling and planning a dental implant procedure can include receiving a patient profile comprising a maxilla or mandible model of the patient and kinematic data associated with the movement of a jaw of the patient, identifying one or more candidate sites for dental implants, generating one or more dental implant parameters, determining an indication of a functional cone, determining one or more dental implant contact points, generating a constraint map, selecting an implant model, and generating a modified model.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

This application claims the benefit under 35 U.S.C. § 119(e) to U.S. Provisional Application No. 63/213,607, entitled “SYSTEMS, METHODS, AND DEVICES FOR AUGMENTED DENTAL IMPLANT SURGERY USING KINEMATIC DATA,” filed Jun. 22, 2021, and the entirety of this application is hereby incorporated by reference herein for all purposes.

### BACKGROUND

### Field

This application relates generally to dental implant surgery.

### Description

Dental implant surgery can involve replacing tooth roots with metal posts and/or replacing damaged or missing teeth with artificial teeth, also called implant caps, that look and function like real teeth to provide a more permanent and/or aesthetically pleasing alternative to dentures or bridges. Planning a surgical procedure to place the implant typically requires the surgeon to account for several variables of the patient's physiology. For example, when selecting the type of implant to be used, its size, depth and/or angular orientation, one may want to consider the patient's bone structure, gumline, surrounding teeth, and/or others.

### SUMMARY

This disclosure describes surgical planning systems (referred to herein as “the system”) and methods for using the same for dental implant surgery. In particular, in some embodiments, and, more specifically, to dental implant surgery enhanced using kinematic data derived from capturing the motion of a patient's jaw. In some embodiments, the system comprises a jaw motion tracking subsystem connected to a computer system configured with surgical planning software. According to some embodiments, the jaw motion tracking system may be connected to a computer system that is networked to a remote computer system configured with surgical planning software. In some embodiments, the jaw motion tracking subsystem comprises a detector, a wearable headset with tracking markers, and software configured to record data from the detector. In some embodiments, the motion tracking system may comprise a detector alone. In some embodiments, the jaw motion tracking system is used to capture a patient's jaw motion (e.g., by recording a video) and use the captured motion to construct kinematic data representing the motion of a patient's jaw. In some embodiments, the constructed kinematic data may be used to render a visual representation of the motion of the patient's jaw to aid the surgeon in selecting and positioning potential implant targets. In some embodiments, the computer system may be further configured to calculate optimization parameters for implant placement, including but not limited to implant depth, angle relative to the bone, size, and/or the like.

In some embodiments, the computer system may be configured to output 3D printable models that can be used to manufacture surgical guides for guided surgery. In some embodiments, the computer system may be connected directly or through a network to a 3D printer. In some embodiments, the computer system may be configured to output a navigated surgery plan for use with a surgical navigation system. In some embodiments, the computer system may be connected directly or through a network to a surgical navigation system. In some embodiments, the system may be compatible with external systems to output a navigated surgery plan.

For purposes of this summary, certain aspects, advantages, and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein.

In some aspects, the techniques described herein relate to a computer-implemented method for oral surgery planning including: receiving, by a computing system, a patient profile, wherein the patient profile includes: patient anatomy data, wherein the patient anatomy data includes one or more models of a maxilla or mandible of the patient; and kinematic data associated with movement of a jaw of the patient; identifying, by the computing system based at least in part on the received patient profile, one or more candidate sites for dental implants; and generating, by the computing system based at least in part on the identified one or more candidate sites and the kinematic data, one or more dental implant parameters.

In some aspects, the techniques described herein relate to a computer-implemented method, wherein the patient profile includes any combination of one or more of bone volume, bone density, relative bone density, location of a nerve, or location of a sinus.

In some aspects, the techniques described herein relate to a computer-implemented method, further including: determining, by the computing system, a proposed crown geometry; determining, by the computing system based at least in part on the kinematic data, an indication of a functional cone; determining, by the computing system based at least in part on the patient profile and the proposed crown geometry, one or more crown contact points; generating, by the computing system based at least in part on the one or more crown contact points, a constraint map; selecting, by the computing system based at least in part on the constraint map, an implant model; and generating, by the computing system based at least in part on the constraint map and the implant model, a modified implant model.

In some aspects, the techniques described herein relate to a computer-implemented method, further including: determining, by the computing system, a proposed crown geometry; automatically determining, by the computing system based at least in part on the kinematic data, an indication of a functional cone; automatically determining, by the computing system based at least in part on the patient profile, one or more crown contact points; and automatically selecting, by the computing system based at least in part on the crown contact points, an implant model.

In some aspects, the techniques described herein relate to a computer-implemented method, wherein generating the modified model includes minimizing one or more stresses on the dental implant.

In some aspects, the techniques described herein relate to a computer-implemented method, wherein identifying one or more candidate sites for dental implants includes comparing the one or more models of the maxilla or mandible of the patient to one or more reference models.

In some aspects, the techniques described herein relate to a computer-implemented method, wherein identifying one or more candidate sites for dental implants includes automatically analyzing a bone of the patient to determine any combination of one or more of: a dental arc, an inter-tooth separation, a bone volume, and a relative bone density.

In some aspects, the techniques described herein relate to a computer-implemented method, wherein the one or more dental implant parameters include any combination of one or more of: a location of the dental implant relative to a bone surface, an implant type, an implant material, a burial depth, an implant angle relative to the bone surface, an implant size, a crown size, and a crown geometry.

In some aspects, the techniques described herein relate to a computer-implemented method, wherein at least one of the crown size and the crown geometry is based at least in part on a prosthetic project, a prosthetic tooth, or an existing tooth of a patient.

In some aspects, the techniques described herein relate to a computer-implemented method, further including: determining, by the computing system based on the patient profile, that one or more candidate sites have insufficient bone volume or insufficient bone density for performing an implant procedure.

In some aspects, the techniques described herein relate to a computer-implemented method, wherein determining one or more dental implant contact points includes determining contact at one or more stages of jaw motion based at least in part on the indication of the functional cone and the patient anatomy, wherein the jaw motion includes recorded motion, simulated motion, or both.

In some aspects, the techniques described herein relate to a computer-implemented method, wherein selecting an implant model includes using an artificial intelligence engine to select a pre-configured model from a model database.

In some aspects, the techniques described herein relate to a computer-implemented method, wherein generating implant parameters includes: providing patient data to an artificial intelligence model, the artificial intelligence model configured to generate implant parameters.

In some aspects, the techniques described herein relate to a computer-implemented method, further including: receiving, by the computing system, an indication of a surgical outcome; and retraining, by the computing system, the artificial intelligence model using the received indication of the surgical outcome.

In some aspects, the techniques described herein relate to a computer-implemented method, further including: providing, to a user, an interface for modifying one or more implant parameters.

In some aspects, the techniques described herein relate to a computer-implemented method, further including: generating a surgical guide, wherein the surgical guide includes a 3D model of a guide that may be used during a surgical procedure.

In some aspects, the techniques described herein relate to a computer-implemented method, further including providing the surgical guide to a 3D printer.

In some aspects, the techniques described herein relate to a computer-implemented method, further including generating a surgical navigation plan.

In some aspects, the techniques described herein relate to a computer-implemented method, further including providing a visualization and interaction interface.

In some aspects, the techniques described herein relate to an oral surgery planning system including: a computing system including: a computer readable storage medium having program instructions embodied therewith; and one or more processors configured to execute the program instructions to cause the computing system to: receive a patient profile, wherein the patient profile includes: patient anatomy data, wherein the patient anatomy data includes one or more models of a maxilla or mandible of the patient; and kinematic data associated with movement of a jaw of the patient; identify, based at least in part on the received patient profile, one or more candidate sites for dental implants; and generate, based at least in part on the identified one or more candidate sites and the kinematic data, one or more dental implant parameters.

In some aspects, the techniques described herein relate to an oral surgery planning system, wherein the patient anatomy data includes one or more models of a maxilla or mandible of the patient.

In some aspects, the techniques described herein relate to an oral surgery planning system, wherein the patient profile includes any combination of one or more of bone volume, bone density, relative bone density, location of a nerve, or location of a sinus.

In some aspects, the techniques described herein relate to an oral surgery planning system, wherein the program instructions further include instructions that, when executed by the one or more processors, cause the computing system to: determine a proposed crown geometry; determine, based at least in part on the kinematic data, an indication of a functional cone; determine, based at least in part on the patient profile and the proposed crown geometry, one or more crown contact points; generate, based at least in part on the one or more crown contact points, a constraint map; select, based at least in part on the constraint map, an implant model; and generate, based at least in part on the constraint map and the implant model, a modified implant model.

In some aspects, the techniques described herein relate to an oral surgery planning system, wherein the program instructions further include instructions that, when executed by the one or more processors, cause the computing system to: determine a proposed crown geometry; automatically determine, based at least in part on the kinematic data, an indication of a functional cone; automatically determine, based at least in part on the patient profile, one or more crown contact points; and automatically select, based at least in part on the crown contact points, an implant model.

In some aspects, the techniques described herein relate to an oral surgery planning system, wherein generating the modified model includes minimizing one or more stresses on the dental implant.

In some aspects, the techniques described herein relate to an oral surgery planning system, wherein identifying one or more candidate sites for dental implants includes comparing the one or more models of the maxilla or mandible of the patient to one or more reference models.

In some aspects, the techniques described herein relate to an oral surgery planning system, wherein identifying one or more candidate sites for dental implants includes automatically analyzing a bone of the patient to determine any combination of one or more of: a dental arc, an inter-tooth separation, a bone volume, and a relative bone density.

In some aspects, the techniques described herein relate to an oral surgery planning system, wherein the one or more dental implant parameters include any combination of one or more of: a location of the dental implant relative to a bone surface, an implant type, an implant material, a burial depth, an implant angle relative to the bone surface, an implant size, a crown size, and a crown geometry.

In some aspects, the techniques described herein relate to an oral surgery planning system, wherein at least one of the crown size and the crown geometry is based at least in part on a prosthetic project, a prosthetic tooth, or an existing tooth of a patient.

In some aspects, the techniques described herein relate to an oral surgery planning system, wherein the program instructions further include instructions that, when executed by the one or more processors, cause the computing system to: determine, based on the patient profile, that one or more candidate sites have insufficient bone volume or insufficient bone density for performing an implant procedure.

In some aspects, the techniques described herein relate to an oral surgery planning system, wherein determining one or more dental implant contact points includes determining contact at one or more stages of jaw motion based at least in part on the indication of the functional cone and the patient anatomy data, wherein the jaw motion includes recorded motion, simulated motion, or both.

In some aspects, the techniques described herein relate to an oral surgery planning system, wherein selecting an implant model includes using an artificial intelligence engine to select a pre-configured model from a model database.

In some aspects, the techniques described herein relate to an oral surgery planning system, wherein the program instructions further include instructions that, when executed by the one or more processors, cause the computing system to: provide patient data to an artificial intelligence model, the artificial intelligence model configured to generate implant parameters.

In some aspects, the techniques described herein relate to an oral surgery planning system, wherein the program instructions further include instructions that, when executed by the one or more processors, cause the computing system to: receive an indication of a surgical outcome; and retrain the artificial intelligence model using the received indication of the surgical outcome.

In some aspects, the techniques described herein relate to an oral surgery planning system, wherein the program instructions further include instructions that, when executed by the one or more processors, cause the computing system to: provide, to a user, an interface for modifying one or more implant parameters.

In some aspects, the techniques described herein relate to an oral surgery planning system, wherein the program instructions further include instructions that, when executed by the one or more processors, cause the computing system to: generate a surgical guide, wherein the surgical guide includes a 3D model of a guide that may be used during a surgical procedure.

In some aspects, the techniques described herein relate to an oral surgery planning system, wherein the program instructions further include instructions that, when executed by the one or more processors, cause the computing system to provide the surgical guide to a 3D printer.

In some aspects, the techniques described herein relate to an oral surgery planning system, wherein the program instructions further include instructions that, when executed by the one or more processors, cause the computing system to generate a surgical navigation plan.

In some aspects, the techniques described herein relate to an oral surgery planning system, wherein the program instructions further include instructions that, when executed by the one or more processors, cause the computing system to provide a visualization and interaction interface.

In some aspects, the techniques described herein relate to an oral surgery planning system, further including: a jaw motion tracking headset; and a jaw motion tracking detector.

All of these embodiments are intended to be within the scope of the invention herein disclosed. These and other embodiments will become readily apparent to those skilled in the art from the following detailed description having reference to the attached figures, the invention not being limited to any particular disclosed embodiment(s).

For purposes of this summary, certain aspects, advantages, and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein.

### BRIEF DESCRIPTION OF THE DRAWINGS

These and other features, aspects, and advantages of the disclosure are described with reference to drawings of certain embodiments, which are intended to illustrate, but not to limit, the present disclosure. It is to be understood that the accompanying drawings, which are incorporated in and constitute a part of this specification, are for the purpose of illustrating concepts disclosed herein and may not be to scale.

FIG. 1A shows an example of implant placement without consideration of a functional cone.

FIG. 1B shows an example of implant placement with consideration of a functional cone according to some embodiments.

FIG. 2 shows a functional cone and the placement of an implant made in consideration of the functional cone according to some embodiments.

FIG. 3A is a schematic diagram illustrating an example embodiment of a jaw motion tracking system and modeling/planning system.

FIG. 3B is a schematic diagram illustrating an example embodiment of a jaw motion tracking system and modeling/planning system.

FIG. 3C is a schematic diagram illustrating an example embodiment of a jaw motion simulation system and modeling/planning system.

FIG. 4A is an example of a tracking headset that may be used for capturing jaw motion according to some embodiments.

FIG. 4B is an example of a tracking camera system according to some embodiments.

FIG. 5 is a schematic diagram illustrating an example embodiment of an automated modeling and planning system for augmented dental implant surgery using kinematic data depicting various components of the system.

FIG. 6 is a schematic diagram illustrating an example embodiment of an implant design module of an automated modeling and planning system for augmented dental implant surgery using kinematic data depicting various components of the system.

FIG. 7A is a schematic diagram illustrating an example embodiment of a system for augmented dental implant surgery using kinematic data depicting various components of the system.

FIG. 7B is a schematic diagram illustrating an example embodiment of a system for augmented dental implant surgery using kinematic data depicting various components of the system operating over a network.

FIG. 8 is a flowchart illustrating an overview of an example embodiment for performing a dental implant surgery augmented using kinematic data.

FIG. 9 is a flowchart illustrating an overview of an example embodiment for training and using an AI engine to provide parametric implant suggestions according to some embodiments herein.

FIG. 10 is a flowchart illustrating a process for training an artificial intelligence or machine learning model according to some embodiments.

FIGS. 11A and 11B are images depicting feature determination according to some embodiments herein.

FIG. 12 is an image depicting feature identification and implant site determination according to some embodiments.

FIG. 13 shows example images and plots of radiodensity according to some embodiments.

FIG. 14 illustrates implant site identification according to some embodiments herein.

FIGS. 15A and 15B illustrate bone centroid identification according to some embodiments.

FIG. 16 illustrates the identification of an implant location according to some embodiments.

FIG. 17 is a diagram illustrating a collaboration platform according to some embodiments.

FIG. 18 is a diagram of an example computer system configured for use with an example embodiment(s) of a system for augmented dental implant surgery using kinematic data.

### DETAILED DESCRIPTION

Although several embodiments, examples, and illustrations are disclosed below, it will be understood by those of ordinary skill in the art that the inventions described herein extend beyond the specifically disclosed embodiments, examples, and illustrations and includes other uses of the inventions and obvious modifications and equivalents thereof. Embodiments of the inventions are described with reference to the accompanying figures, wherein like numerals refer to like elements throughout. The terminology used in the description presented herein is not intended to be interpreted in any limited or restrictive manner simply because it is being used in conjunction with a detailed description of some specific embodiments of the inventions. In addition, embodiments of the inventions can comprise several novel features and no single feature is solely responsible for its desirable attributes or is essential to practicing the inventions herein described.

As discussed above, some embodiments described herein are directed to automating dental implant surgery using kinematic data describing the patient's jaw motion. For example, in some embodiments, the system can be configured to utilize kinematic data derived from capturing the motion of a jaw of a patient to provide enhanced dental implant surgery. Without use or consideration of kinematic data, in some embodiments, the surgeon may have to rely on experience and guesswork in judging the appropriate parameters for an implant, which may result in sub-optimal surgical outcomes; this may lead to breakage or chipping of caps or crowns, bone resorption around the implant or post (e.g., due to transverse forces), breakage of the abutment and/or the connection between the implant and the abutment, discomfort or adverse health events for the patient, or aesthetically displeasing tooth geometry. In such instances, the surgeon may also require more time to identify parameters for an implant that fit the patient's needs.

Some embodiments of the systems, methods, and devices described herein are directed at addressing these technical shortcomings. In particular, in some embodiments described herein, surgeons may take advantage of automated calculation and generation of implant parameters based on kinematic data derived from a patient's jaw motion (which may be captured and/or simulated) to optimize the implant parameters to both save time and achieve better outcomes.

In some embodiments, an implant may be a manufactured replacement for a missing tooth comprising a post that is affixed or inserted into a patient's jaw bone. In some embodiments, a cap or crown mimicking the appearance of a tooth that may be permanently attached to the implant. In some embodiments, kinematic data may be captured by using one or more detectors to record the motion of a patient's jaw. In some embodiments, the motion data may be recorded while the patient is wearing one or more visual markers to be used in translating the video into kinematic data. In some embodiments, the one or more detectors may be attached to a computer configured to accept the kinematic data and import it into one or more surgical planning software packages. In some embodiments, the kinematic data may be used to render a visual representation of the motion of the patient's jaw to aid the surgeon in selecting and positioning potential implant targets. In some embodiments, the kinematic data may be further used to calculate a functional cone. The functional cone represents an envelope whose limits have been determined by the displacement of a point during mandibular movements. The mandibular movements may be a patient's actual, recorded movements, may be generated by simulating the masticatory system as a mechanical system, or both. The functional cone may represent the average angles and stresses involved in the movement of the patient's jaw.

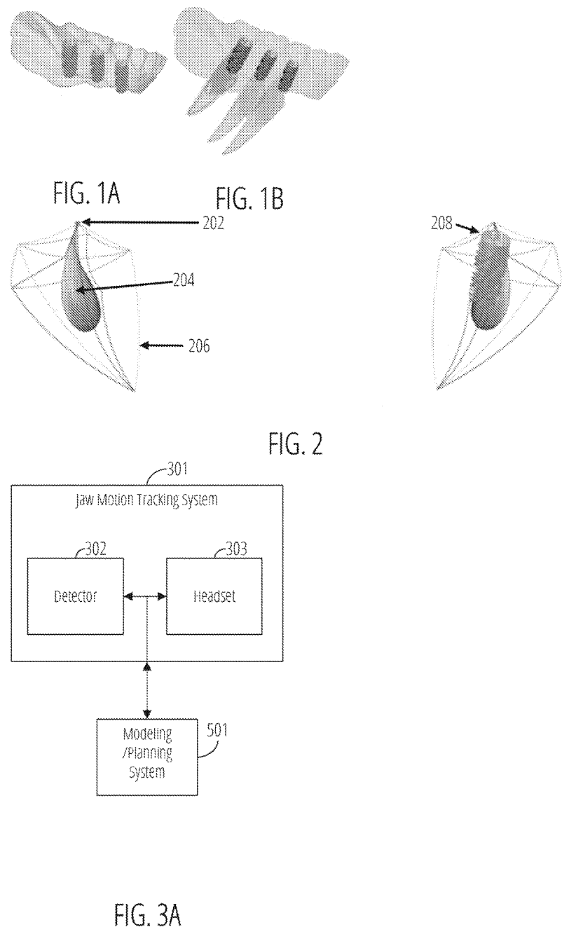

FIG. 1A illustrates an example of implant placement when the functional cone is not taken into consideration. For example, implants may be placed at non-ideal angles which can have several undesirable results as described herein. FIG. 1B shows an example of implants that have been positioned with consideration of the functional cone. The implants in FIG. 1B can have several advantages over those in 1A as discussed herein.

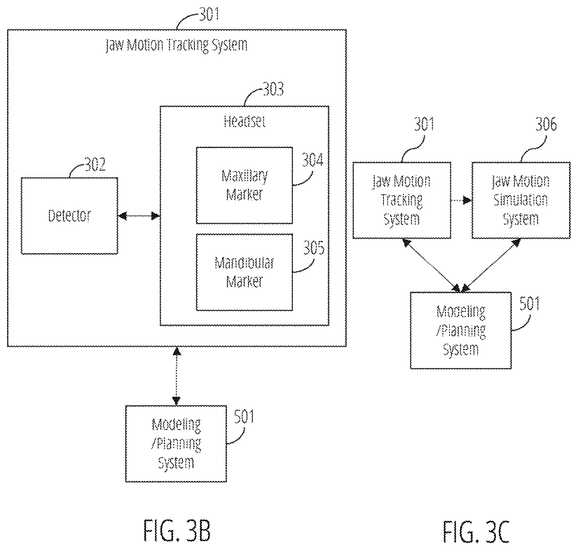

FIG. 2 shows a functional cone and the placement of an implant made in consideration of the functional cone according to some embodiments. As illustrated in FIG. 2, a functional cone has an emergence point 202, a 3D envelope of functional movement 204, and a 3D envelope of border movement 206. An implant 208 can be placed close to the centroid of the functional cone. The 3D envelope of functional movement 204 may correspond to the range of movements of a patient's jaw during normal functional actions. The 3D envelope of border movement may correspond to a maximum range of jaw movement.

In some embodiments, the computer system may be further configured to automatically calculate optimal parameters for implant placement, including but not limited to implant depth, angle, size, and/or type. In some embodiments, the functional cone may be used in these calculations to, inter alia, determine placement parameters that resist occlusal loads, disperse stresses in the surrounding bone, avoid excess bone resorption, apply uniform (or close to uniform) stresses on the connections between the abutment and the implant, and avoid fractures of the crown, abutment, and so forth. For example, the functional cone can be used to determine an angle that minimizes shear stress on an implant, minimize stress on surrounding bone tissue, and/or select an appropriate implant type and/or geometry. In these embodiments, an implant site may be further determined based on applying the functional cone to the point of a sited implant's emergency from the patient's jaws.

As discussed herein, “dental implant” is defined to include any type of dental implant, including for posts, implants, dental bridges, dental splints, crowns, dentures, or any other dental fixture. As discussed herein, “dental implant surgery” is defined to include any treatment or procedure for planning, developing, modeling, preparing, creating, inserting, and/or attaching any of the aforementioned dental implants.

### Jaw Motion Tracking

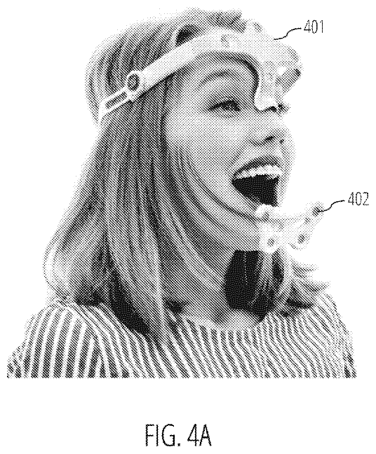

As shown in FIG. 3A, in some embodiments a system may comprise a jaw motion tracking system 301, which may further comprise a detector 302 (e.g., a jaw motion tracking detector) and a motion tracking headset 303. In some embodiments, the detector can be connected to a modeling and planning system 501. In some embodiments, the detector may be connected to the modeling and planning system via a data transfer cable, a network cable, or a wireless network. The detector may be any device or combination of devices capable of recording the movement over time of a patient's mandible relative to their maxilla such as, for example, a camera without depth-sensing capabilities. In some embodiments, the camera system may be capable of depth sensing. For example, a camera system may use stereoscopy, structural light, time of flight, light detection and ranging (LIDAR), or other depth sensing principles. As shown in FIG. 3B, in some embodiments, the motion tracking headset may further comprise a maxillary marker 304 and/or a mandibular marker 305. In some embodiments, the maxillary and/or mandibular markers further comprise fiducial markers that may be used to track the relative motion of a patient's mandible and/or maxilla. In some embodiments, instead of or in addition to optical detection systems, an inertial measurement unit can be used for motion tracking. For example, accelerometers, magnetometers, gyroscopes, and so forth can be used to monitor movements. In some embodiments, an inertial measurement unit can be a microelectromechanical system (MEMS) device.

In some embodiments, instead of a jaw motion tracking system, or in addition to using a jaw motion tracking system, a jaw motion simulation system can be used to simulate the movements of a patient's jaw, for example by treated the masticatory system as a mechanical system. FIG. 3C depicts an example embodiment in which a jaw motion simulation system 306 is in communication with a modeling and planning system 501. In some embodiments, the jaw motion simulation system 306 can receive data from jaw motion tracking system 301, for example condylar points, Bennet angle, and so forth, which can be used in simulating jaw motion of the patient. In some embodiments, the modeling and planning system can use only the jaw motion tracking system data, only the jaw motion simulation system data, or a combination of data from both the jaw motion tracking system and the jaw motion simulation system.

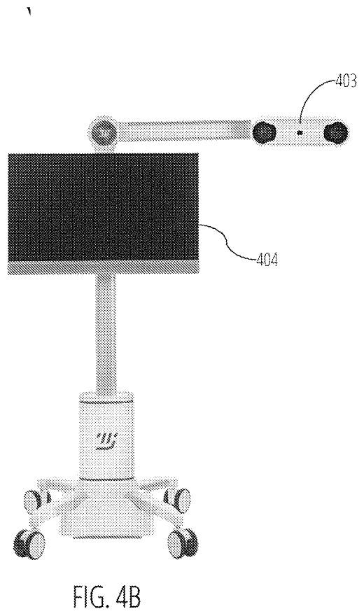

In some embodiments, the maxillary marker 304 may comprise any device which can be affixed, connected, or related to the patient's maxilla that can enable the detector to track the movement of the patient's maxilla. In some embodiments, the mandibular marker 305 may comprise any device which can be affixed, connected, or related to the patient's mandible that can enable the detector to track the movement of the patient's mandible. It will be appreciated that other configurations are possible, which may use additional or different markers. FIG. 4A depicts an example embodiment of a tracking device that comprises a tiara 401 that is affixed to the patient's head and a mandibular marker 402 that is affixed to and that moves with the patient's mandible. In some embodiments, a headset may include one or more sensors that enable the headset to track motion directly (e.g., without the use of an external detector). In some embodiments, an external detector and one or more sensors included in the headset may be used together to track the movements of the patient. FIG. 4B illustrates motion capture device that can be used to capture patient motion, for example by detecting motion of the tiara 401 and mandibular marker 402 of FIG. 4A. The motion capture device can include a stereo camera system 403 configured to capture 3D motion. The motion capture device can, in some embodiments, include a display 404. The display 404 can be used to monitor a motion capture procedure.

### Modeling and Planning System

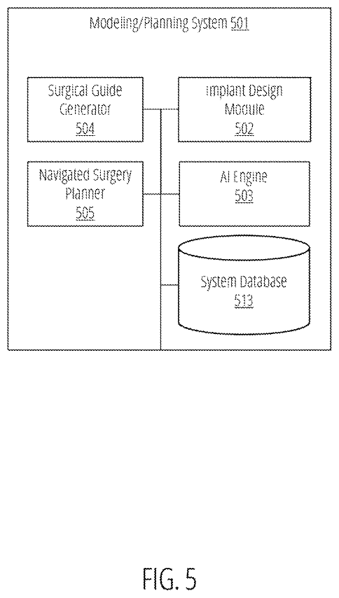

As shown in FIG. 5, in some embodiments the modeling and planning system 501 may comprise an implant design module 502, an AI engine 503, a surgical guide generator 504, a navigated surgery planner 505, and/or a system database 513. In some embodiments, the modeling and planning system comprises software configured to run on a computer system.

In some embodiments, the AI engine 503 can comprise a machine learning algorithm, one or more neural networks, a heuristic engine, and/or a stochastic model.

In some embodiments, the surgical guide generator 504 may be configured to output 3D models representing one or more surgical guides. In some embodiments, a surgical guide may be a device that attaches to the mouth of a patient during surgery to aid the surgeon in maneuvering one or more tools and/or the implant to improve surgical outcomes. In some embodiments, the 3D models may be configured to be manufacturable on-site or by a manufacturer using one or more manufacturing and/or 3D printing technologies. For example, in some embodiments, the modeling and planning system 501 may be connected to a 3D printer and/or milling machine, while in other embodiments, 3D models may be transferred to another system for production, for example using a network file transfer protocol, an application programming interface, e-mail, and so forth.

In some embodiments, the navigated surgery planner 505 may be configured to output one or more surgical navigation plans. In some embodiments, the surgical navigation plan may comprise data compatible with one or more surgical navigation systems. In some embodiments, in order to generate a surgical navigation plan, the navigated surgery planner may be configured to transmit data to an external navigated surgery planning system. The one or more surgical navigation systems may comprise a computerized system that may include sensors and/or indicators to aid the surgeon in guiding one or more tools and/or the implant during surgery to improve surgical outcomes.

In some embodiments, the implant design module 502 may be configured to allow surgeons and/or medical staff to manually or automatically generate dental implant parameters, and to design and/or reconfigure the parameters.

In some embodiments, the system database 513 may comprise a database engine configured to store one or more patient profiles, system settings, and/or usage information. In some embodiments, the one or more patient profiles may each comprise a patient's medical history, models and/or medical images of the patient's anatomy, such as dental X-rays, which may include data describing their existing natural teeth, existing prosthetic teeth, virtual prosthetic project, jaw, nerves, bones, and/or other features. In some embodiments, each patient profile may further comprise data relating to planned surgeries, surgical guide models, and/or surgical navigation plans generated by the system. In some embodiments, the system settings may comprise settings related to the operation of the graphical user interface, connections to external services, and/or device settings. In some embodiments, usage information may comprise statistics relating to use of the software such as logins, access to patient data, and/or log information describing various system activity. In some embodiments, the usage information may further comprise settings and data related to access control, which may comprise user login and password information, access rights to patient data, and/or audit information describing user activity on the system.

In some embodiments, images and/or models of a patient's face may be imported into the modeling and planning system 501 and displayed in conjunction with other patient data and models, for example in order to provide context for the patient and/or surgeon or medical personnel.

### Implant Design Module

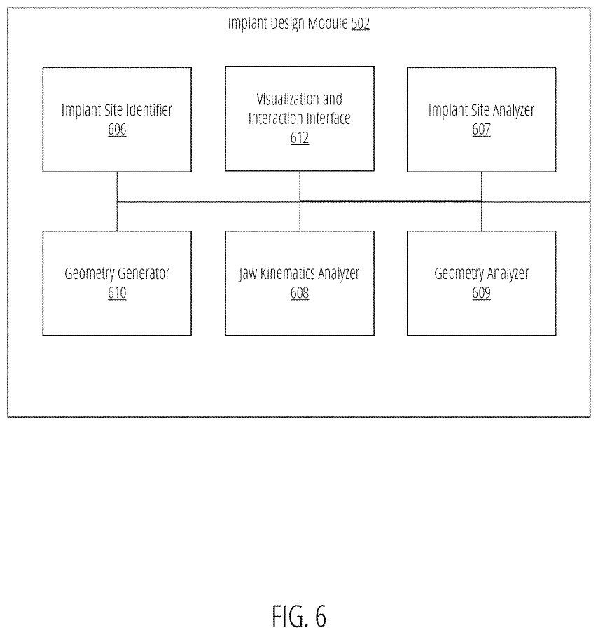

As shown in FIG. 6, in some embodiments the implant design module 502 may comprise an implant site identifier 606, an implant site analyzer 607, a jaw kinematics analyzer 608, an implant geometry analyzer 609, an implant geometry generator 610, and/or a visualization and interaction interface 612. In some embodiments, the implant design module may be used to automatically generate implant parameters based on a patient profile and/or kinematic data constructed from the patient's jaw motion. In some embodiments, a user may select one or more parameters to generate automatically. In some embodiments, the inputs that may be used to generate the parameters can be customized by the user.

In some embodiments, the implant site identifier 606 may comprise a set of algorithms configured to receive one or more models of the patient's maxilla and/or mandible and identify one or more candidate sites for dental implants. In some embodiments, the one or more models may comprise a 3D image of the patient's anatomy acquired by X-ray computed tomography (CT) and/or other medical imaging modalities. In some embodiments, identification of implant sites may be based on a comparison of the models of the patient's maxilla and/or mandible to one or more stored models of human anatomy to identify missing teeth. Some embodiments may further analyze additional data to determine implant sites, such as x-ray data from Cone Beam Computerized Tomography (CBCT), for example to analyze radiodensity (for example, using linear attenuation coefficients, which can be represented in Hounsfield units). In some embodiments, the implant site identifier 606 can differentiate between areas of relatively high and low density. In some embodiments, the implant site identifier 606 can determine and/or apply one or more density thresholds that can be used to differentiate between bone segments that are suitable for implantation and bone segments that are not. In some embodiments, identification of implant sites may be based on automated bone analysis methods. In some embodiments, the implant site identifier may perform a 3D reconstruction of an implant site to facilitate calculations of the patient's bone volume and density. In these embodiments, an implant site may be determined to maximize average bone density surrounding the implant. In some embodiments, the implant site identifier may not compute absolute bone density values. In some embodiments, the implant site identifier may compare relative bone densities. For example, the implant site identifier may compare a region of relatively high density (e.g., a tooth root) to a region of relatively low density (e.g., a jaw bone).

In some embodiments, the identification of potential implant sites may be presented to a user via the visualization and interaction interface 612, and the user may remove, add to, or edit the one or more identified implant sites. In some embodiments, the identified implant sites are stored as part of a patient profile.

In some embodiments, the implant site analyzer 607 may comprise a set of algorithms configured to receive one or more models of the patient's maxilla and/or mandible and a set of implant sites. The implant site analyzer can use various methods for analyzing potential implant sites. These methods may include, for example, automated methods for calculating dental arcs, defining inter-tooth separations, etc. In some embodiments, the implant site analyzer 607 may be further configured to generate, for each of the one or more implant sites, parameters for a dental implant. In some embodiments, the implant parameters may comprise one or more of the implant location relative to the patient's bone surface, implant type, implant material, burial depth, implant angle relative to the bone surface, implant size, crown size, and/or the geometry of the implant cap. In some embodiments, generating implant parameters may further comprise identifying the need for a bone graft to support an implant based on, for example, an insufficiency of local bone volume and/or density.

In some embodiments, the generation of implant parameters may include consideration of one or more anatomical features of the patient. In some embodiments, these anatomical features may include the volume of bone around the implant site, the bone width, quality and density, the height of the patient's gumline above the bone, the location of the patient's sinus, nerves and mental foramen, and/or the like; in some embodiments, an optimal emergence point through the gum can be obtained, taking into account one or more of these parameters. The emergence point can be the intersection between the axis of the implant, characterized by a straight line, with the bone surface or the gingival surface, corresponding to a connection zone of a prosthetic device (e.g., crown) with its implant. In some embodiments, the generation of the implant parameters may further consider biomechanical parameters of the patient's jaw. In some embodiments, the biomechanical parameters may comprise kinematic data describing the patient's jaw motion. In some embodiments, this kinematic data may be received in the form of a functional cone. In some embodiments, the biomechanical behavior of the bone may be used to anticipate stresses on the bone.

In some embodiments, the jaw kinematics analyzer 608 may comprise a set of algorithms configured to receive raw jaw motion data from the jaw motion tracking system. In these embodiments, the jaw kinematics analyzer 608 may analyze the raw data and output a functional cone. In some embodiments, the functional cone can comprise a plurality of displacement vectors. In some embodiments, the functional cone may comprise a set of data describing the maximum range of motion of a patient's jaw. In some embodiments, the functional cone may further comprise a set of vectors describing the stresses that may be generated by a patient's jaw at various positions. For example, the displacement of the mandible is the result of the contraction and/or relaxation of various muscles oriented along multiple axes. Because the motion, displacement vectors, and forces generated by muscles are known or can be approximated, the jaw kinematics analyzer can determine the magnitudes and/or directions of stresses generated on the bone. In some cases, the analyzer can determine stresses on the implant and/or the abutment, for example when their biomechanical behavior is known or can be approximated. Advantageously, the implant can absorb forces with reduced risk of damage if the implant is located at a centroid of the movement. In some embodiments, one or more parts of the functional cone may be provided to the jaw kinematics analyzer 608 as input, and the analyzer may proceed to derive the rest of the functional cone from the provided data.

In some embodiments, the implant geometry analyzer 609 may comprise a set of algorithms configured to receive a functional cone, a model of the patient's mandible and/or maxilla, and/or a proposed crown geometry. In some embodiments, the implant geometry analyzer 609 may be further configured to generate a map of contacts between the proposed implant crown and other features of the patient (e.g., other teeth, crowns, implants, etc.) at various stages of jaw motion based on the patient's anatomy and the functional cone. In some embodiments, the implant geometry analyzer 609 may be further configured to generate a map of the stresses that occur at each of the previously described contact points based on the functional cone, hereinafter referred to as a constraint map. In some embodiments, the constraint map may be generated relative to one or more volumes of space encompassing one or more implant sites. In some embodiments, the implant geometry analyzer may be configured to follow a decision tree in generating the implant parameters. For example, a decision tree process flow could begin by determining boundary conditions such as the locations of nerves, bones, and so forth, and may then narrow implant parameters based on additional information (e.g., bone density, stresses, etc.). In some embodiments, one or more portions of the generated data may be submitted to the AI engine 503 for modification. In some embodiments, one or more portions of the generated data may be initially generated by the AI engine 503.

In some embodiments, the implant geometry generator 610 may comprise a set of algorithms configured to generate one or more models representing the geometry of a dental implant. In some embodiments, the generation of the one or more models may be based on a constraint map generated by the implant geometry analyzer. In some embodiments, the generation of the one or more models may be further configured to generate models that minimize shear stresses on the implant generated by the motion of the patient's jaw. In some embodiments, the generation of the one or more models may be further configured to generate models that minimize the stresses on certain portions of the patient's jaw. In some embodiments, the models may be generated by modifying one or more preconfigured implant models stored in the system database 513. In some embodiments, selection of the one or more preconfigured implant models may be performed by the AI engine 503. In some embodiments, the selection of the one or more preconfigured implant models may be performed by the surgeon or medical personnel or may be performed automatically by the implant geometry generator 610. In some embodiments, the selection of the one or more preconfigured implant models may be performed according to one or more rules stored in the system database 513.

In some embodiments, the visualization and interaction interface 612 comprises a graphical user interface. The graphical user interface may further comprise a visualization window that may display 2D and/or 3D images and/or text. The visualization window may be further configured to display data representing anatomical features, implant geometry, jaw motion paths, statistical data, and/or patient information, for example prior to a dental procedure, during a dental procedure, and/or after a dental procedure. In some embodiments, the graphical user interface may be further configured to be interactive, allowing users to manipulate the displayed data. In some embodiments, the enabled manipulation may include editing the parameters and geometry of implants, for example for planning purposes. In some embodiments, the graphical user interface may be further configured to allow users to generate and/or export images and videos based on the data presented.

### Integrated System for Augmented Dental Implant Surgery Using Kinematic Data

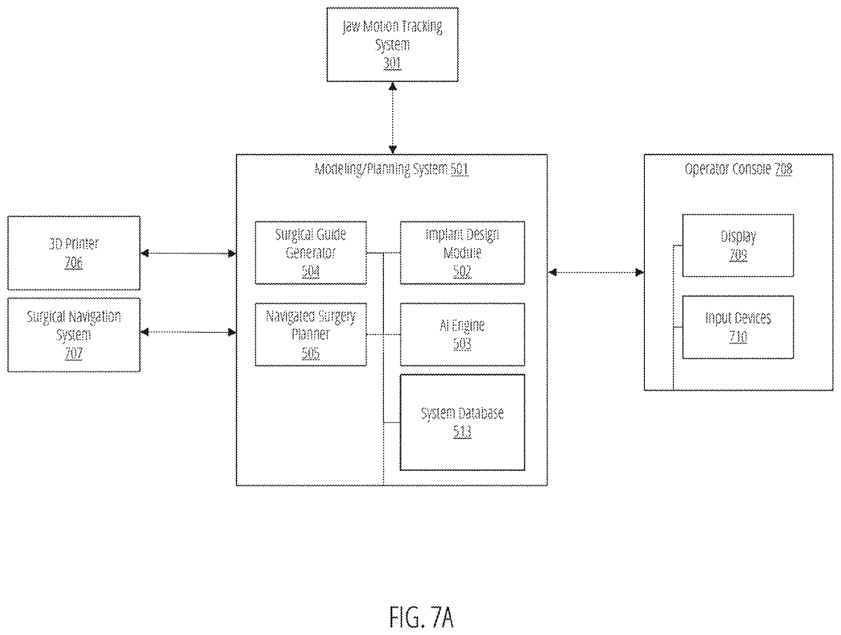

As shown in FIG. 7A, in some embodiments the system may comprise a jaw motion tracking system 301, a modeling and planning system 501, an operator console 708, a 3D printer 706 and/or a surgical navigation system 707.

In some embodiments, the jaw motion tracking system 301 may be configured to record the motion of a patient's jaw and transmit the data to the modeling and planning system 501. In some embodiments, the jaw motion tracking system 301 may analyze and/or format the data before transmitting it to the modeling and planning system. In some embodiments, the jaw motion tracking system may store the data or transmit the data to a storage system for later consumption.

In some embodiments, the modeling and planning system 501 may be a computer system configured to receive motion tracking data from the jaw motion tracking system 301 and convert the motion tracking data into kinematic data. In some embodiments, the modeling and planning system 501 may be further configured to receive one or more patient profiles as input. In some embodiments, the modeling and planning system 501 may be further configured to automatically determine the parameters of one or more dental implants that may be surgically implanted in the patient. In some embodiments, the modeling and planning system 501 may be connected to a 3D printer 706 and may be further configurable to output 3D printable surgical guide models that may be printed on the 3D printer. In some embodiments, additional or alternative computer aided manufacturing hardware may be connected to the modeling and planning system 501, such as milling equipment. In some embodiments, the modeling and planning system may be connected to a surgical navigation system 707 and/or may be further configurable to output one or more surgical navigation plans that may be utilized by the surgical navigation system.

In some embodiments, the operator console 708 may be a computer system configured to provide a user interface for surgeons and/or surgical staff to interact with the modeling and planning system 501. In some embodiments, the operator console can include a display 709 and/or one or more input devices 710. In some embodiments, the modeling and planning system 501 and the operator console 708 may comprise a single computer system. In some embodiments, the operator console 708 may comprise a thin client or other computing device that may interact with the modeling and planning system 501 via a network.

In some embodiments, the 3D printer 706 may be connected to the modeling and planning system 501. In some embodiments, the 3D printer 706 may be configured to use stereolithography, digital light processing, fused deposition modeling, selective laser sintering, multi jet fusion, polyjet, direct metal laser sintering, and/or electron beam melting. In some embodiments, the modeling and planning system may be configured to interact with a connected milling machine.

### Distributed System for Augmented Dental Implant Surgery Using Kinematic Data

As shown in FIG. 7B, in some embodiments the system may comprise a jaw motion tracking system 301, an operator console 708, a 3D printer 706, a surgical navigation system 707, an AI engine 503, and/or a modeling and planning system 501. In some embodiments, the modeling and planning system 501 may be connected to the other modules via one or more computer networks. The computer networks 721, 722, 723, and 724 may comprise wireless and/or wired networks.

### Method for Automating Dental Implant Surgery Using Kinematic Data

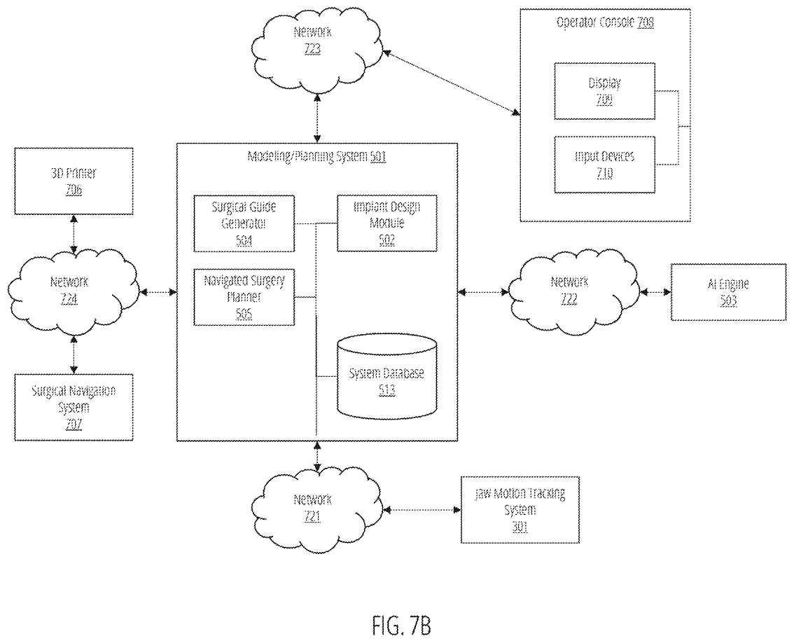

FIG. 8 is a flowchart illustrating an overview of an example embodiment(s) of performing a dental implant surgery augmented using kinematic data. In some embodiments, the process may deviate from that shown in FIG. 8. Some embodiments may include more steps, fewer steps, and/or steps may be performed in a different order than shown. As shown in FIG. 8,

In some embodiments, a surgeon or medical personnel may capture static physical data about the patient at block 802. In some embodiments, a surgeon or medical personnel may attach the mandibular marker 305 and maxillary marker 304 and/or headset 303 to a patient at block 804. The static data may include x-ray from CBCT or CT scans, 3D models of teeth from an intraoral scanner or lab scanner, and so forth.

In some embodiments, a detector can identify the location of fiducial markers attached to the patient at block 806, and/or capture motion image data at block 808. In some embodiments, the surgeon or medical personnel may instruct the patient to move their jaw during the capture process.

In some embodiments, the jaw motion tracking system tracks the patient's jaw movement based on the captured image data and transfers it to the modeling and planning system at block 810. Alternatively or additionally, the patient's jaw movement may be simulated, for example by treating the masticatory system as a mechanical system. In some embodiments, the simulated movement can be customized based at least in part on recorded patient motion. For example, the recorded motion may have various flaws or be incomplete, but may still be useful for determining some parameters such as condylar slop, Bennett angle, and so forth, which can help improve the accuracy of simulated movement.

In some embodiments, the modeling and planning system 501 may store the kinematic data based on the captured jaw movement as part of the patient profile at block 812. In some embodiments, the modeling and planning system 501 may further identify the implant target and analyze it in conjunction with the captured kinematic data.

In some embodiments, static physical data may be accessed at block 814 and the kinematic data, patient profile, and static data may be used to analyze kinematic data and/or to identify an implant target at block 816. The data can be analyzed by the AI engine 503 at block 818, which can include receiving data from and/or providing data to model database 820. The AI analysis may then be utilized by the modeling and planning system 501 to automatically generate implant size, type, and/or placement parameters at block 822.

In some embodiments, the surgeon and/or surgical staff may have the choice of accepting or modifying the implant parameters suggested by the modeling and planning system 501 at block 824. If the surgeon and/or surgical staff rejects the suggested parameters at 824, the surgeon and/or surgical staff can modify the implant design at block 826. In some embodiments, the AI engine 503 can receive the modifications and retrain an AI model. For example, the AI engine 503 may determine which parameter or parameters were modified (e.g., emergence point, bone volume, stress map, placement of the implant with respect to the functional cone, and so forth). For example, different providers can have different preferences, and the AI engine 503 can adapt to those preferences.

In some embodiments, the modeling and planning system 501 may be further configured to generate a surgical guide model at block 828 and/or to generate a surgical navigation plan at block 830.

In some embodiments, one or more surgical guides may be manufactured based on the previously generated surgical guide model at block 832. In some embodiments, one or more implants may be selected and/or manufactured based on the previously established parameters at block 834. Alternatively, in some embodiments an existing implant matching the previously established parameters may be selected from an implant library.

In some embodiments, the surgeon may perform the implant surgery with the aid of the generated surgical guide and/or navigation plan at block 836. In some embodiments, the surgeon may record postoperative outcomes at block 838.

In some embodiments, the AI engine 503 may be further configured to update its internal models based on the patient profile and post-operative data based on the surgical outcomes at block 840, which may include data about implant failures.

### AI Engine for Automating Parametric Implants

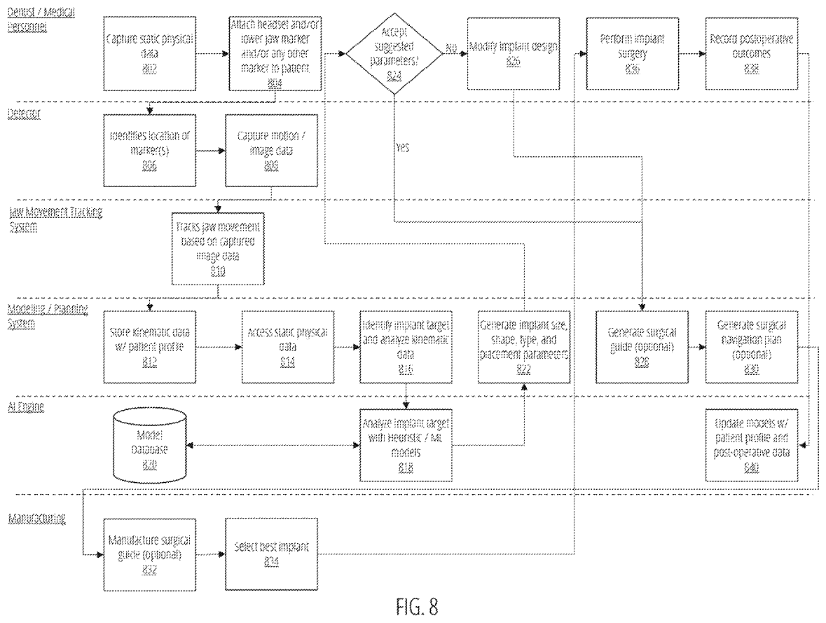

FIG. 9 is a flowchart illustrating an overview of an example embodiment(s) of training and using an AI engine to provide parametric implant suggestions. In some embodiments, the AI engine 503 may comprise one or more analysis algorithms and a model database 820. In some embodiments, the model database 820 can contain one or more pre-configured models. In some embodiments, the system developer or provider may collect training data at block 902 to perform the initial training of the AI engine 503 at block 904. In some embodiments, the training data may comprise data about past surgeries in the form of pairs of implant and patient parameters with corresponding surgical outcome data. In some embodiments, the implant parameters may comprise one or more of the implant location relative to the patient's bone surface, implant type, implant material, burial depth, implant angle relative to the bone surface, implant size (e.g., length, diameter), crown size, and/or crown geometry. In some embodiments, the patient parameters may comprise mandibular and/or maxillary bone geometry, bone quality, bone density, nerve and mental foramen location, sinus location, jaw kinematic data and/or functional cone, and/or biographic information such as, for example, age, sex, and/or medical history. In some embodiments, the surgical outcome data may comprise one or more perioperative complications, postoperative longevity of the implant, adverse events related to the implant, and/or patient satisfaction.

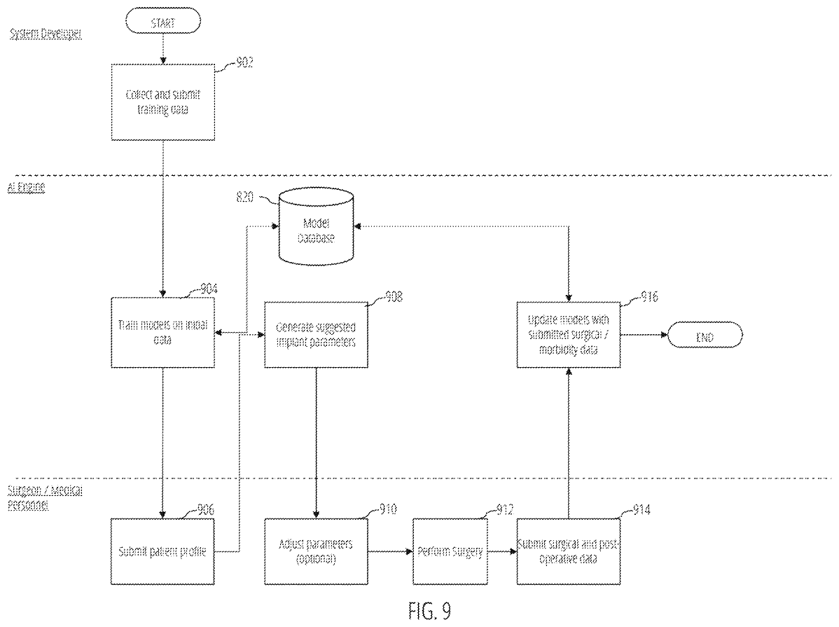

FIG. 10 depicts a flow chart for training an artificial intelligence or machine learning model according to some embodiments. In some embodiments, the training step 904 of FIG. 9 may implement a process similar to or the same as the training process 1000 depicted in FIG. 10. At block 1001, the system may receive a dataset that includes various information such as patient profile information, jaw motion information, and so forth. At block 1002, one or more transformations may be performed on the data. For example, data may require transformations to conform to expected input formats, for example to conform with expected date formatting, to conform to a particular tooth numbering system (e.g., Universal Numbering System, FDI World Dental Federation notation, or Palmer notation). In some embodiments, the data may undergo conversions to prepare it for use in training an AI or ML algorithm, which typically operates using data that has undergone some form of normalization or other alteration. For example, categorical data may be encoded in a particular manner. Nominal data may be encoded using one-hot encoding, binary encoding, feature hashing, or other suitable encoding methods. Ordinal data may be encoded using ordinal encoding, polynomial encoding, Helmert encoding, and so forth. Numerical data may be normalized, for example by scaling data to a maximum of 1 and a minimum of 0 or −1. At block 1003, the system may create, from the received dataset, training, tuning, and testing/validation datasets. The training dataset 1004 may be used during training to determine variables for forming a predictive model. The tuning dataset 1005 may be used to select final models and to prevent or correct overfitting that may occur during training with the training dataset 1004, as the trained model should be generally applicable to a broad spectrum of patients, rather than to the particularities of the training data set (for example, if the training data set is biased towards patients with relatively high or low bone density, wide or narrow dental arches, etc.). The testing dataset 1006 may be used after training and tuning to evaluate the model. For example, the testing dataset 1006 may be used to check if the model is overfitted to the training dataset. The system, in training loop 1014, may train the model at 1007 using the training dataset 1004. Training may be conducted in a supervised, unsupervised, or partially supervised manner. At block 1008, the system may evaluate the model according to one or more evaluation criteria. For example, the evaluation may include determining how often implant suggestions would be suitable for a patient based on a variety of criteria such as contact points, shear stresses, bone density at the implant site, and so forth. At block 1009, the system may determine if the model meets the one or more evaluation criteria. If the model fails evaluation, the system may, at block 1010, tune the model using the tuning dataset 1005, repeating the training 1007 and evaluation 1008 until the model passes the evaluation at block 1009. Once the model passes the evaluation at 1009, the system may exit the model training loop 1014. The testing dataset 1006 may be run through the trained model 1011 and, at block 1012, the system may evaluate the results. If the evaluation fails, at block 1013, the system may reenter training loop 1014 for additional training and tuning. If the model passes, the system may stop the training process, resulting in a trained model 1011. In some embodiments, the training process may be modified. For example, the system may not use a testing dataset 1006 in some embodiments. In some embodiments, the system may use a single dataset. In some embodiments, the system may use two datasets. In some embodiments, the system may use more than three datasets.

Returning to FIG. 9, in some embodiments, the surgeon and/or medical personnel may submit a patient's profile to an AI engine (e.g., the AI engine 503) at block 906. In some embodiments, the patient profile may comprise the patient's mandibular and maxillary bone geometry, bone quality, bone density, nerve and mental foramen location, sinus location, jaw kinematic data or functional cone, tooth positions, occlusal architecture, and/or biographic information such as for example age, sex, and/or surgical history. In some embodiments, the patient profile may include other parameters not specified herein.

In some embodiments, the patient profile may be analyzed by the AI engine 503 at block 908, and the AI engine 503 may then generate suggested implant parameters. In some embodiments, the AI analysis may be configured to analyze a subset of the patient profile. In some embodiments, the subset may comprise one or more of the patient's mandibular and maxillary bone geometry, bone quality, bone density, nerve and mental foramen location, sinus location, jaw kinematic data or functional cone, biographic information, and/or surgical history. In some embodiments, a subset of said data may be used. For example, in some embodiments, only a portion of the jaw kinematic data may be used. For example, in some embodiments less data may be used in order to reduce computational needs, or some data may be excluded, such as jaw kinematic data representing extreme movements that the patient would not ordinarily make. In some embodiments, the AI engine may analyze all or part of an existing prosthesis, a prosthetic project (e.g., a simulation or a future shape of a prosthetic tooth), and so forth. In some embodiments, implants may be placed under virtually-designed teeth. In some embodiments, the surgeon and/or medical personnel can adjust the suggested parameters at block 910, and the surgeon may perform the surgery at block 912 based on the suggested parameters.

In some embodiments, after surgery, the surgeon and/or medical personnel may submit surgical and post-operative data to the AI engine 503 at block 914. In some embodiments, the surgical and/or post-operative data may comprise a list of perioperative complications, post-operative longevity of the implant, adverse events related to the implant, and/or patient satisfaction. In some embodiments, the submitted data may be used by the AI engine 503 to update the models in the model database 820 at block 916.

As shown in FIG. 7B, in some embodiments the AI engine 503 may be connected to the modeling and planning system via a network. In some embodiments, the AI engine 503 may be hosted by a commercial service provider. In some embodiments, the AI engine 503 may be software configured to run on a cloud computing platform. In some embodiments, the AI engine 503 may be trained on a first computing system and deployed on the first computing system. In some embodiments, the AI engine 503 may be trained on a first computing system and deployed on a second computing system. For example, it may be beneficial to have a large amount of computing resources during model training, but such computing power may not be needed for deployment. Similarly, network bandwidth may be relatively unimportant during training but more important in deployment so that the AI engine 503 is able to handle incoming requests.

### Example Methods of Automating Implant Surgery

The systems and methods described herein may be used to automate various aspects of dental treatment and planning, including simple and complex treatment scenarios. In some embodiments, a system can be configured to determine the eventual placement of teeth by proposing implant positions of one or more missing teeth. This can include capturing kinematic data, capturing intra-oral scan information to determine the shapes and placement of a patient's existing teeth and bone structures, and so forth. In some cases, the patient may not have missing teeth, but may instead need to replace one or more existing teeth, in which case information may be captured relating the shape, structure, and/or positioning of the patient's current teeth so that a suitable replacement may be formed. The system may determine positioning of an implant and propose guides to aid in performing surgery and/or plans for performing navigated surgery.

In some embodiments, a patient may have existing prosthetics, and an implant may be used for placing and/or securing the prosthetics. In some cases, the implant area may be located in front of or behind a prosthetic tooth. In such cases, a process for automatically determining placement of an implant can include taking into account information such as the tooth centroid, bone volume, bone density, and/or functional cone. The implant area may, in some cases, be identified using automated modeling and/or segmentation of the patient's existing prosthetic and/or natural teeth. In some cases, a patient may have a removable prosthesis which can be replaced with a fixed prosthesis. The shape of the existing prosthesis can be to define the location of the implant.

In some embodiments, a patient may not have existing prosthetics and/or may be edentulous. In such cases, a more complex approach may be used as there is reduced or eliminated information about existing (natural or prosthetic) teeth. In some embodiments, a method may comprise identifying a dental arch, developing a dental implantation plan, determining a zone of volume for implantation, performing bone reconstruction, and identifying a functional tooth centroid (which may be determined from a functional cone). The functional cone may be centered at an emergence point of the implant, and implant dimensions (e.g., diameter, length, width, height, and so forth) may be determined and a planned implant or work area may be identified.

In some embodiments, an implant and/or crown may be chosen from one or more libraries. For example, libraries may comprise implants from different manufacturers. In some embodiments, a provider (e.g., a dentist) may select a manufacturer and an algorithm may then be used to select the appropriate diameter and/or length of the implant based at least in part on a combination of one or more of bone volume, prosthesis location, emergence point, functional cone, and so forth.

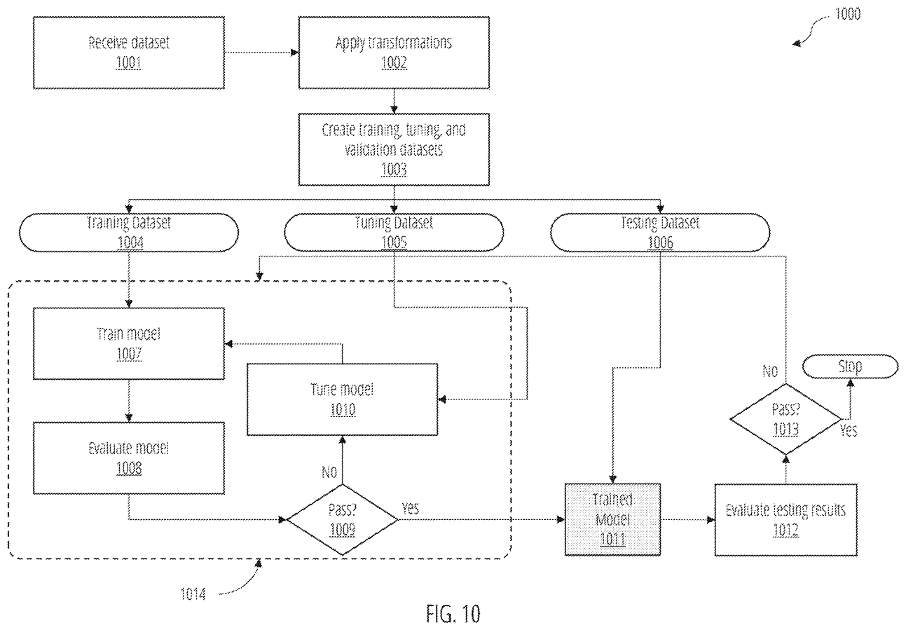

In some embodiments, identifying a dental arch may be performed by a system using a 3D model of the arches, a CBCT scan, x-ray, or other image. In some embodiments, CBCT images may contain artifacts that render them unsuitable or non-ideal for identifying a dental arch. FIG. 11A illustrates the identification of a dental arch 1101. In some embodiments, a tooth crown may be identified by, in part, reorienting at least one model according along the occlusal plane as depicted in FIG. 11B, showing a crown 1102.

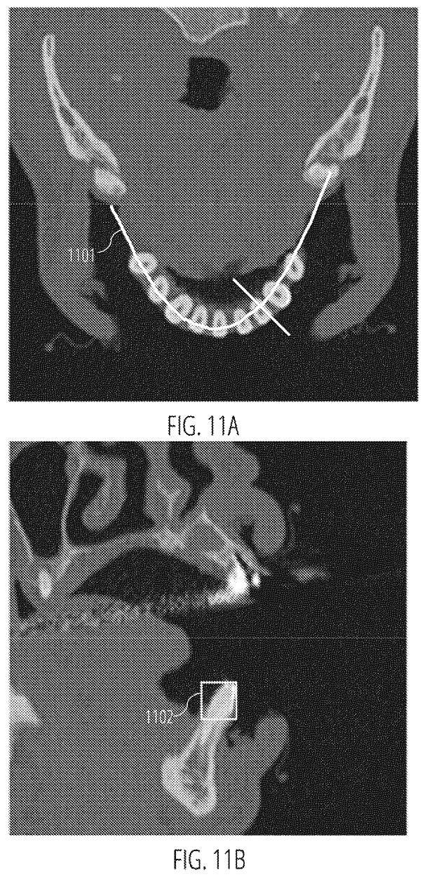

From an image such as FIG. 11A, various features can be determined, as illustrated in FIG. 12. For example, the image may include teeth 1201, missing teeth areas 1202, and dental interstices 1203. In some embodiments, multiple images may be used or multiple measurements may be carried out to determine an average dental arc.

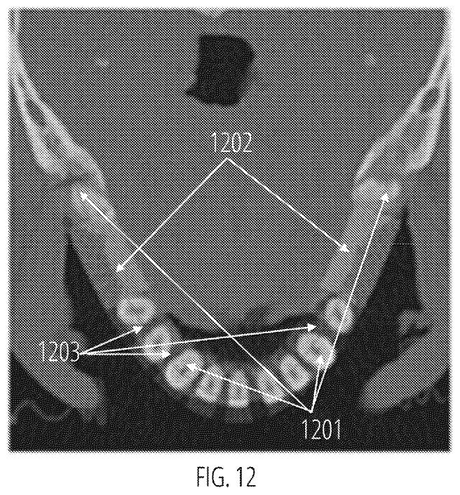

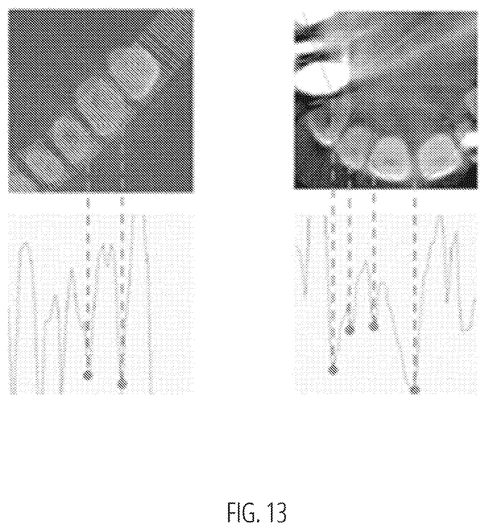

As shown in FIG. 13, once an arc has been identified, cross-sections perpendicular to the arc may be analyzed to optimize location determinations and identify an ideal rea. For example, the gray level on an x-ray or CT scan may indicate radiodensity, and different materials (e.g., bone, roots, soft tissue, and so forth) may have differing radiodensities which may, in some embodiments, be measured in Hounsfield Units. In some embodiments, radiodensity data may help to identify an implant area, as illustrated in FIG. 14. For example, a system may be configured to use imaging data to determine bone density, to determine bone volume, to determine tooth locations, border areas between teeth, and so forth. In some embodiments, bone volume calculations may be used to determine the positioning center of an implant, by for example selecting an area with relatively high bone volume. In some embodiments, choosing an area with relatively large bone volume may not lead to an ideal implant result. For example, instead, an area of relatively high bone density may be selected. In some embodiments, the system may determine that a bone graft or the other procedure would be beneficial based on a determination that the patient lacks an area with suitable bone volume and density to perform an implant procedure.

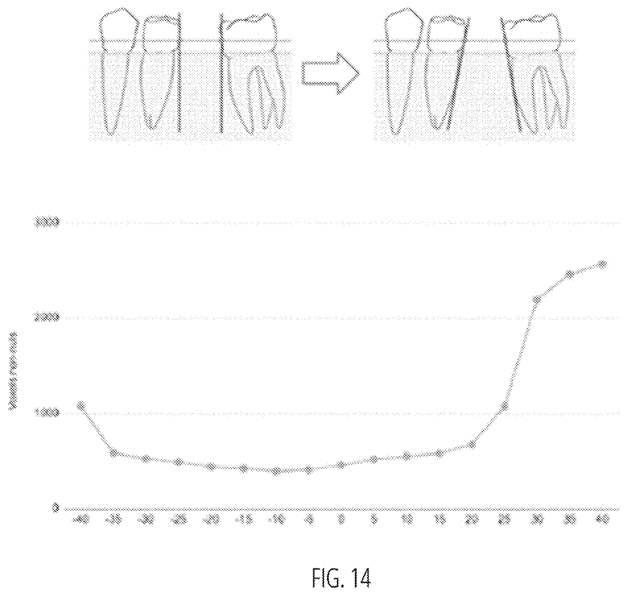

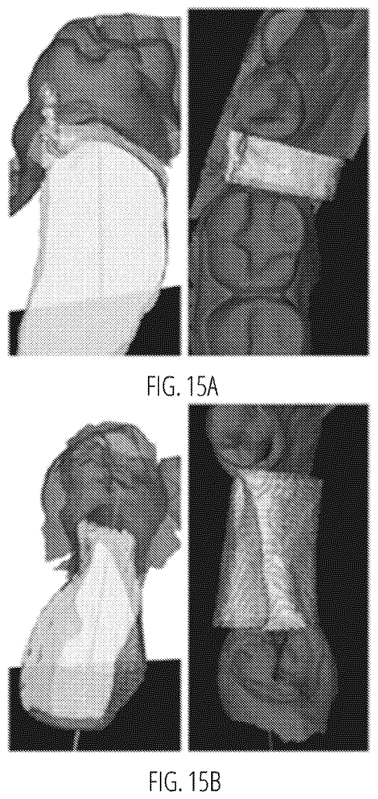

In some embodiments, the system may identify a bone centroid which may be the center of the bone volume, as illustrated in FIGS. 15A and 15B. In the absence of a tooth, the bone centroid can enable the system to compensate for the absence of the tooth in the dental arch. In some embodiments, the system may consider the centroid and the top of the ridge to correct an axis for placing an implant and to obtain an emergence point as illustrated in FIG. 16. Accordingly, the system can enable a surgeon to improve planning by taking into account biomechanical data and bone density information. In some embodiments, the bone centroid may enable the determination of a functional dental centroid that considers the dental arch.

In some embodiments, the system may calculate a functional cone starting with the emergence point and applying movement (e.g., kinematic data captured from the patient). Thus, the implant may be oriented at least in part by taking the functional cone into account.

In some embodiments, implants may be selected according to the prosthetic project and even without teeth, the use of the dental arch may already serve as a prosthetic project.

### Collaboration Platform

In some embodiments, for example as illustrated in FIG. 7A, a dentist may have access to equipment on-site for preparing surgical guides. However, in some cases, dentists may not have access to such equipment and thus may outsource manufacturing to a third party. Similarly, dentists will often use outside laboratories to provide implants, crowns, and so forth. Thus, it would be advantageous for dentists and other providers to be able to easily interact with laboratories to facilitate the preparation of implants, caps, surgical guides, and so forth.

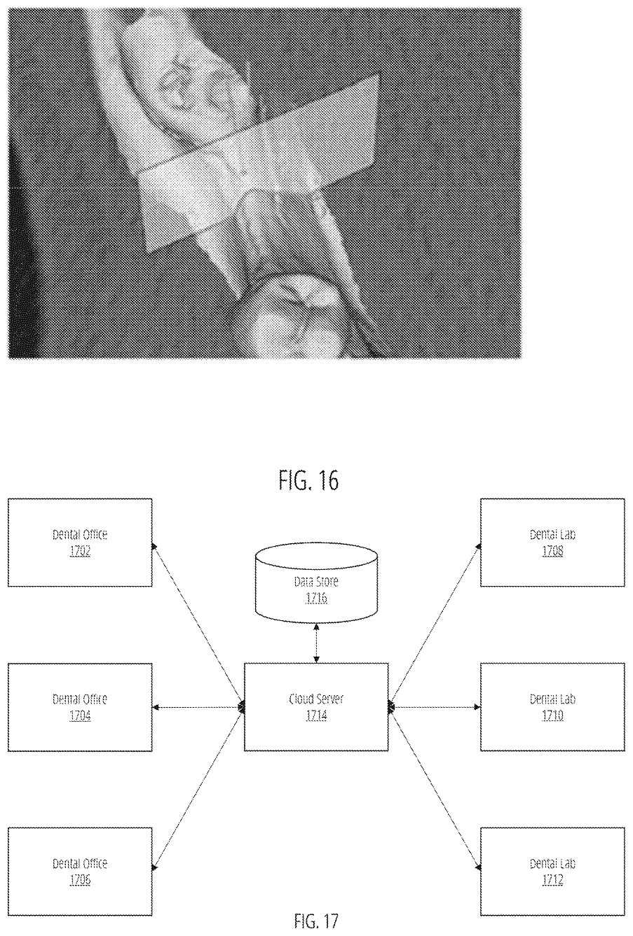

FIG. 17 illustrates an example embodiment of a collaboration platform to enable communication among providers and laboratories. For example, as depicted in FIG. 17, dental offices 1702, 1704, and 1706, and dental labs 1708, 1710, and 1712 can be in communication with a cloud server 1714. The cloud server 1714 can be in communication with a data store 1716. The cloud server 1714 can enable the dental offices 1702, 1704, 1706 to communicate with the dental labs 1708, 1710, 1712. For example, the dental offices can send data to the cloud server 1714 where it can be stored in data store 1716 and can be accessed by the dental labs. In some embodiments, the dental labs may provide data to the cloud server 1714 that can then be accessed by one or more of the dental offices. In some embodiments, the dental offices and/or dental labs can communicate using smartphones, tablets, desktop computers, laptop computers, or other devices. For example, in some embodiments, equipment used for collecting patient data and/or planning procedures may have capabilities for communicating over a network.

In some embodiments, dental offices may provide relevant parameters and information that dental labs can use to prepare implants, crowns, and so forth for a particular procedure. Dental labs can provide information that dental offices may find useful, for example available implant depths and diameters, available materials, available crown shapes and sizes, and so forth. In some embodiments, dental offices may provide information about the patient such as name, contact information, insurance information, billing information, and so forth to the dental labs, for example if the dental lab bills directly to the patient and/or the patient's insurance.

In some embodiments, different dental offices may use the platform depicted in FIG. 17 to collaborate with each other. For example, dental office 1702 may wish to consult with dental offices 1704 and 1706 regarding a procedure. Using the platform, the dental office 1702 can make some or all of the relevant patient data available to the other dental offices.

In some embodiments, the collaboration platform can include various features for protecting data and patient privacy. For example, in some embodiments, information may be stored in the data store 1716 in an encrypted format. In some embodiments, the collaboration platform may have a user permission system that enables users of the platform to control access to information. For example, users of the platform may give access to some users but not to other users, or a user may wish to allow another user to temporarily access information (for example, for consulting with another dentist, or when one dentist or provider is filling in for another provider).

In some embodiments, users of the platform may upload information for processing by the platform. For example, storing kinematic data, storing patient profiles, storing static physical data, identifying implant targets, analyzing kinematic data, generating implant parameters, generating surgical guides, generating surgical navigation plans, analyzing implant parameters, and so forth can all be run on the platform. In some embodiments, some portions of a dental planning process may be run locally on a provider's own systems while other portions may be run remotely. For example, computationally intensive tasks such as running and/or training machine learning models may be offloaded to a cloud server.

### Computer System

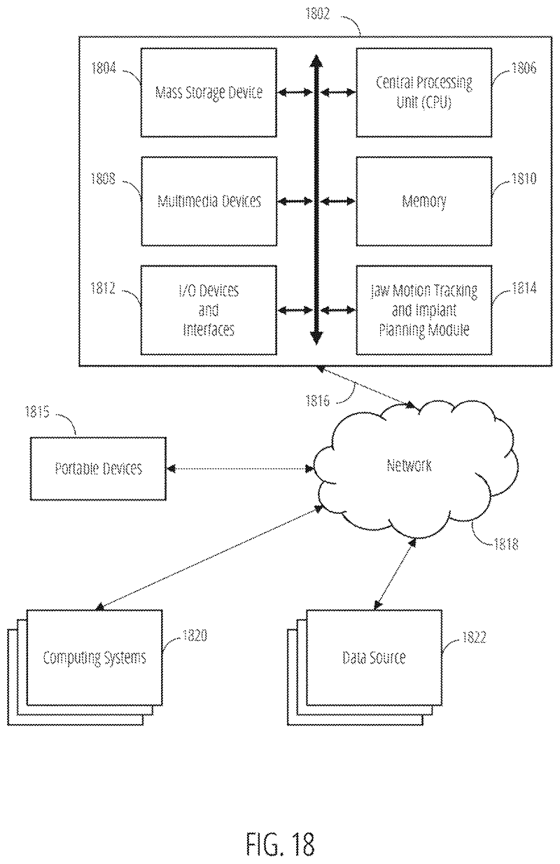

In some embodiments, the systems, processes, and methods described herein may be implemented using one or more computing systems, such as the one illustrated in FIG. 18. The example computer system 1802 is in communication with one or more computing systems 1820, one or more portable devices 1815, and/or one or more data sources 1822 via one or more networks 1818. While FIG. 14 illustrates an embodiment of a computing system 1802, it is recognized that the functionality provided for in the components and modules of computer system 1802 may be combined into fewer components and modules, or further separated into additional components and modules.

The computer system 1802 can comprise a Jaw Motion Tracking and/or Implant Planning module 1814 that carries out the functions, methods, acts, and/or processes described herein. The Jaw Motion Tracking and Implant Planning module 1814 is executed on the computer system 1802 by a central processing unit 1806 discussed further below.

In general, the word “module,” as used herein, refers to logic embodied in hardware or firmware or to a collection of software instructions, having entry and exit points. Modules are written in a programming language, such as JAVA, C or C++, Python, or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 1802 includes one or more processing units (CPU) 1806, which may comprise a microprocessor. The computer system 1802 further includes a physical memory 1810, such as random access memory (RAM) for temporary storage of information, a read only memory (ROM) for permanent storage of information, and a mass storage device 1804, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 1802 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 1802 includes one or more input/output (I/O) devices and interfaces 1812, such as a keyboard, mouse, touch pad, and printer. The I/O devices and interfaces 1812 can include one or more display devices, such as a monitor, that allows the visual presentation of data to a participant. More particularly, a display device provides for the presentation of GUIs as application software data, and multi-media presentations, for example. The I/O devices and interfaces 1812 can also provide a communications interface to various external devices. The computer system 1802 may comprise one or more multi-media devices 1808, such as speakers, video cards, graphics accelerators, and microphones, for example.

The computer system 1802 may run on a variety of computing devices, such as a server, a Windows server, a Structure Query Language server, a Unix Server, a personal computer, a laptop computer, and so forth. In other embodiments, the computer system 1802 may run on a cluster computer system, a mainframe computer system and/or other computing system suitable for controlling and/or communicating with large databases, performing high volume transaction processing, and generating reports from large databases. The computing system 1802 is generally controlled and coordinated by an operating system software, such as z/OS, Windows, Linux, UNIX, BSD, SunOS, Solaris, MacOS, or other compatible operating systems, including proprietary operating systems. Operating systems control and schedule computer processes for execution, perform memory management, provide file system, networking, and I/O services, and provide a user interface, such as a graphical user interface (GUI), among other things.

The computer system 1802 illustrated in FIG. 18 is coupled to a network 1818, such as a LAN, WAN, or the Internet via a communication link 1816 (wired, wireless, or a combination thereof). Network 1818 communicates with various computing devices and/or other electronic devices. Network 1818 is communicating with one or more computing systems 1820, one or more portable devices 1815, and/or one or more data sources 1822. The Jaw Motion Tracking and Implant Planning module 1814 may access or may be accessed by computing systems 1820 and/or data sources 1822 through a web-enabled user access point. Connections may be a direct physical connection, a virtual connection, and other connection type. The web-enabled user access point may comprise a browser module that uses text, graphics, audio, video, and other media to present data and to allow interaction with data via the network 1818.

Access to the Jaw Motion Tracking and Implant Planning module 1814 of the computer system 1802 by computing systems 1820, portable devices 1815, and/or by data sources 1822 may be through a web-enabled user access point such as the computing systems 1820, portable devices 1815, and/or data sources 1822 personal computer, cellular phone, smartphone, laptop, tablet computer, e-reader device, audio player, or other device capable of connecting to the network 1818. Such a device may have a browser module that is implemented as a module that uses text, graphics, audio, video, and other media to present data and to allow interaction with data via the network 1818.

The output module may be implemented as a combination of an all-points addressable display such as a cathode ray tube (CRT), a liquid crystal display (LCD), a plasma display, or other types and/or combinations of displays. The output module may be implemented to communicate with input devices 1812 and they also include software with the appropriate interfaces which allow a user to access data through the use of stylized screen elements, such as menus, windows, dialogue boxes, toolbars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 1802 may comprise a physical or logical connection established between a remote microprocessor and a mainframe host computer for the express purpose of uploading, downloading, or viewing interactive data and databases on-line in real time. The remote microprocessor may be operated by an entity operating the computer system 1802, including the client server systems or the main server system, and/or may be operated by one or more of the data sources 1822, the portable devices 1815, and/or the computing systems 1820. In some embodiments, terminal emulation software may be used on the microprocessor for participating in the micro-mainframe link.

In some embodiments, computing systems 1820 who are internal to an entity operating the computer system 1802 may access the Jaw Motion Tracking and Implant Planning module 1814 internally as an application or process run by the CPU 1806.

The computing system 1802 may include one or more internal and/or external data sources (for example, data sources 1822). In some embodiments, one or more of the data repositories and the data sources described above may be implemented using a relational database, such as DB2, Sybase, Oracle, CodeBase, and Microsoft® SQL Server as well as other types of databases such as a flat-file database, an entity relationship database, and object-oriented database, and/or a record-based database.

The computer system 1802 may also access one or more databases 1822. The databases 1822 may be stored in a database or data repository. The computer system 1802 may access the one or more databases 1822 through a network 1818 or may directly access the database or data repository through I/O devices and interfaces 1812. The data repository storing the one or more databases 1822 may reside within the computer system 1802.

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

### Other Embodiments

Although this invention has been disclosed in the context of some embodiments and examples, it will be understood by those skilled in the art that the invention extends beyond the specifically disclosed embodiments to other alternative embodiments and/or uses of the invention and obvious modifications and equivalents thereof. In addition, while several variations of the embodiments of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the embodiments of the disclosed invention. Any methods disclosed herein need not be performed in the order recited. Thus, it is intended that the scope of the invention herein disclosed should not be limited by the particular embodiments described above.

Conditional language, such as, among others, “can,” “could,” “might,” or “may,” unless specifically stated otherwise, or otherwise understood within the context as used, is generally intended to convey that some embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment. The headings used herein are for the convenience of the reader only and are not meant to limit the scope of the inventions or claims.

Further, while the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (e.g., as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present.

1. A computer-implemented method for oral surgery planning comprising:
receiving, by a computing system, a patient profile, wherein the patient profile comprises:
patient anatomy data, wherein the patient anatomy data comprises one or more models of a maxilla or mandible of the patient; and
kinematic data associated with movement of a jaw of the patient;
identifying, by the computing system based at least in part on the received patient profile, one or more candidate sites for dental implants; and
generating, by the computing system based at least in part on the identified one or more candidate sites and the kinematic data, one or more dental implant parameters.

2. The computer-implemented method of claim 1, wherein the patient profile includes any combination of one or more of bone volume, bone density, relative bone density, location of a nerve, or location of a sinus.

3. The computer-implemented method of claim 1, further comprising:
determining, by the computing system, a proposed crown geometry;
determining, by the computing system based at least in part on the kinematic data, an indication of a functional cone;
determining, by the computing system based at least in part on the patient profile and the proposed crown geometry, one or more crown contact points;
generating, by the computing system based at least in part on the one or more crown contact points, a constraint map;
selecting, by the computing system based at least in part on the constraint map, an implant model; and
generating, by the computing system based at least in part on the constraint map and the implant model, a modified implant model.

4. The computer-implemented method of claim 1, further comprising:
determining, by the computing system, a proposed crown geometry;
automatically determining, by the computing system based at least in part on the kinematic data, an indication of a functional cone;
automatically determining, by the computing system based at least in part on the patient profile, one or more crown contact points; and
automatically selecting, by the computing system based at least in part on the crown contact points, an implant model.

5. The computer-implemented method of claim 3, wherein generating the modified model comprises minimizing one or more stresses on the dental implant.

6. The computer-implemented method of claim 1, wherein identifying one or more candidate sites for dental implants comprises comparing the one or more models of the maxilla or mandible of the patient to one or more reference models.

7. The computer-implemented method of claim 1, wherein identifying one or more candidate sites for dental implants comprises automatically analyzing a bone of the patient to determine any combination of one or more of: a dental arc, an inter-tooth separation, a bone volume, and a relative bone density.

8. The computer-implemented method of claim 1, wherein the one or more dental implant parameters comprise any combination of one or more of: a location of the dental implant relative to a bone surface, an implant type, an implant material, a burial depth, an implant angle relative to the bone surface, an implant size, a crown size, and a crown geometry.

9. The computer-implemented method of claim 8, wherein at least one of the crown size and the crown geometry is based at least in part on a prosthetic project, a prosthetic tooth, or an existing tooth of a patient.

10. The computer-implemented method of claim 1, further comprising:
determining, by the computing system based on the patient profile, that one or more candidate sites have insufficient bone volume or insufficient bone density for performing an implant procedure.

11. The computer-implemented method of claim 4, wherein determining one or more dental implant contact points comprises determining contact at one or more stages of jaw motion based at least in part on the indication of the functional cone and the patient anatomy, wherein the jaw motion comprises recorded motion, simulated motion, or both.

12. The computer-implemented method of claim 1, wherein selecting an implant model comprises using an artificial intelligence engine to select a pre-configured model from a model database.

13. The computer-implemented method of claim 1, wherein generating implant parameters comprises:
providing patient data to an artificial intelligence model, the artificial intelligence model configured to generate implant parameters.

14. The computer-implemented method of claim 13, further comprising:
receiving, by the computing system, an indication of a surgical outcome; and
retraining, by the computing system, the artificial intelligence model using the received indication of the surgical outcome.

15. The computer-implemented method of claim 1, further comprising:
providing, to a user, an interface for modifying one or more implant parameters.

16. The computer-implemented method of claim 1, further comprising:
generating a surgical guide, wherein the surgical guide comprises a 3D model of a guide that may be used during a surgical procedure.

17. (canceled)

18. The computer-implemented method of claim 1, further comprising generating a surgical navigation plan.

19. The computer-implemented method of claim 1, further comprising providing a visualization and interaction interface.

20. An oral surgery planning system comprising:
a computing system comprising:
a computer readable storage medium having program instructions embodied therewith; and
one or more processors configured to execute the program instructions to cause the computing system to:
receive a patient profile, wherein the patient profile comprises:
patient anatomy data, wherein the patient anatomy data comprises one or more models of a maxilla or mandible of the patient; and
kinematic data associated with movement of a jaw of the patient;
identify, based at least in part on the received patient profile, one or more candidate sites for dental implants; and
generate, based at least in part on the identified one or more candidate sites and the kinematic data, one or more dental implant parameters.

21-39. (canceled)

40. The oral surgery planning system of claim 20, further comprising:
a jaw motion tracking headset; and
a jaw motion tracking detector.
